(19) United States
(12) Patent Application Publication
(10) Pub. No.: US 2025/0388554 A1
(43) Pub. Date: Dec. 25, 2025

(54) ANTIBACTERIAL COMPOUNDS, METHOD OF PRODUCTION AND USE THEREOF

(71) Applicant: NATIONAL INSTITUE OF IMMUNOLOGY, New Delhi (IN)

(72) Inventor: Bichitra Kumar Biswal, New Delhi (IN)

(21) Appl. No.: 18/748,576

(22) Filed: Jun. 20, 2024

(30) Foreign Application Priority Data
Jun. 19, 2024 (SG) 10202401792P

(51) Int. Cl. C07D 249/08; A61K 31/4196; A61K 45/06; A61P 31/06; C07D 249/12; C07D 249/14; C07D 403/12

(52) U.S. Cl. CPC C07D 249/08; A61K 31/4196; A61K 45/06; A61P 31/06; C07D 249/12; C07D 249/14; C07D 403/12

(57) ABSTRACT

The present invention discloses an antibacterial compound of Formula 1 or pharmaceutically acceptable salt thereof:

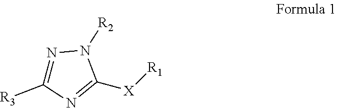

wherein X is CH, S, CH—NH₂;
R₁ is C1-C8 alkyl, substituted alkyl, alkyl amine, substituted amine; preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

The Formula 1 or pharmaceutically acceptable salt thereof is an imidazole glycerol phosphate dehydratase (IGPD) inhibitor and treats or prevents or ameliorates tuberculosis.

### FIELD OF THE INVENTION

The present invention relates particularly but not exclusively to antibacterial compounds. More specifically, the present invention relates to compounds of Formula 1, methods of treating or preventing bacterial infections in a subject using a compound of Formula 1 and using said compound as a growth inhibitor of Mycobacterium tuberculosis.

### BACKGROUND

Human tuberculosis (TB), caused by the bacterial pathogen Mycobacterium tuberculosis (Mtb) is a major health issue worldwide. To combat Mtb pathogenesis the current drugs target the cell wall and protein biosynthesis of Mtb. However, the biggest challenge faced in ameliorating Mtb infection is that the drugs develop resistance against these biological processes around which the drugs have been designed. Thus, it is required that new prospects for potential drug targets are studied against Mtb infection. It has been found from the current studies that amino acids biosynthesis by Mtb play major roles in mounting sustained TB infections. The studies show that Mtb manages its His requirement through de novo histidine biosynthesis pathway, even when the host strategy in defence against the infection is to starve Mtb of Histidine. This suggests that disrupting the function of this pathway may curtail bacterial growth and therefore may represent a new way of limiting TB infection. Furthermore, the fundamental requirement of His for Mtb viability and the inability of humans to not make His de novo make this pathway an attractive drug target.

Imidazoleglycerol-phosphate dehydratase (IGPD), catalyses the conversion of imidazoleglycerol-phosphate (IGP) to imidazoleacetol-phosphate in the histidine biosynthesis pathway. The said enzyme catalyses de novo synthesis of His in plants and microorganisms, however it is absent in mammals. Thus, IGPD belongs to a prime category of anti-bacterial, anti-herbs and anti-fungal drug target as it is absent in mammals. The IGPD of Mtb is one of the key enzymes in the histidine biosynthesis pathway and has been recognized as the potentially underexploited drug target for anti-tuberculosis treatment. The present invention provides a target-based approach, to identify anti-IGPD inhibitors showing chemical inhibition of the Mtb de novo histidine biosynthesis leading to a significant bacterial clearance.

Targeting Mtb His pathway is particularly advantageous as the path is critical for Mtb to mount a sustained TB infection in vivo. Since it is absent in humans, pathway enzymes lack human homologs. This implies that developing inhibitors of these enzymes would minimize the potential of cross-reactivity.

### SUMMARY OF THE INVENTION

As will be realized in the following description, the invention is capable of other and different embodiments and its several details are capable of modifications in various respects, all without departing from the scope of the present invention. This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In one aspect, there is provided an antibacterial compound of Formula 1 or pharmaceutically acceptable salt thereof;

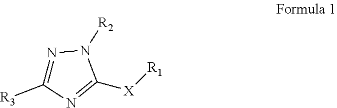

wherein X is CH, S, CH—NH₂;
R₁ is C1-C8 alkyl, substituted alkyl, alkyl amine, substituted amine; preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine,N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

In a further aspect, there is provided a compound of Formula 1 or pharmaceutically acceptable salt, solvate, or hydrate thereof as a growth inhibitor of Mycobacterium tuberculosis;

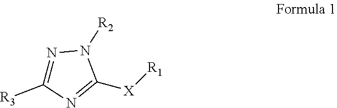

wherein X is CH, S, CH—NH₂;
R₁ is C1-C8 alkyl, substituted alkyl, alkyl amine, substituted amine; preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropylethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

In another aspect, there is provided a compound of Formula 1 or pharmaceutically acceptable salt, solvate, or hydrate thereof as an imidazole glycerol phosphate dehydratase (IGPD) inhibitor for use in treatment or prevention or amelioration of tuberculosis.

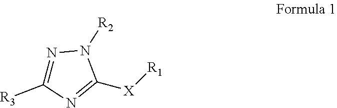

wherein X is CH, S, CH—NH₂;
R1 is C1-C8 alkyl, substituted alkyl, alkyl amine, substituted amine; preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine,N1-ethyl-N1-propylethane-1,2-diamine,cyclobutyamine, phosphate, sulphate
R2 is hydrogen, alkyl, substituted alkyl; preferably R2 is methyl, propyl, isopropyl;
R3 is hydrogen, alkyl, substituted alkyl; and preferably R3 is methyl, propyl, isopropyl.

In a further aspect, a pharmaceutical composition comprising an effective amount of compound with Formula 1 or pharmaceutically acceptable salt thereof, together with at least one pharmaceutically acceptable carrier, diluent, excipient, and/or adjuvant is provided

In another embodiment, there is provided a method for inhibiting Mycobacterium tuberculosis, wherein said method comprises administering a therapeutically effective amount of a compound with Formula 1 to a subject in need thereof.

### BRIEF DESCRIPTION OF THE DRAWINGS

This invention will be described by way of non-limiting embodiments of the present invention, with reference to the accompanying drawings, in which:

FIG. 1A illustrates Electron density maps of the X-ray crystal structures of enzyme/inhibitor complex SF1.

FIG. 1B illustrates Electron density maps of the X-ray crystal structures of enzyme/inhibitor complex SF2.

FIG. 1C illustrates Electron density maps of the X-ray crystal structures of enzyme/inhibitor complex SF3.

FIG. 2A illustrates interactions of IGPD with SF1 inhibitors.

FIG. 2B illustrates interactions of IGPD with SF2 inhibitor.

FIG. 2C illustrates interactions of IGPD with SF3 inhibitor.

FIG. 3A illustrates activity of compounds SF1, SF2 and SF3 against the enzyme IGPD.

FIG. 3B illustrates MIC values against H37Rv and drug resistant clinical isolates (S6 & S7).

FIG. 3C illustrates growth curve analysis showing the effect of SF1, SF2, and SF3 on Mtb growth at a concentration of 1×MIC for 14 days with and without histidine.

FIG. 4A illustrates efficacy testing in Mtb infected macrophage model for SF1.

FIG. 4B illustrates efficacy testing in Mtb infected macrophage model for SF2.

FIG. 5A illustrates animal toxicity data showing biochemical parameters with compound SF2.

FIG. 5B illustrates animal toxicity data showing biochemical parameters with compound SF3.

FIG. 6A illustrates the schematic method to study clearance of Mtb upon administration of test compounds SF1, SF2 (1×), SF2 (2×) and SF3.

FIG. 6B illustrates the mice lungs bacterial burden post administration of test compounds SF1, SF2 (1×), SF2 (2×) and SF3 orally at indicated time points.

FIG. 6C illustrates the mice spleen bacterial burden post administration of test compounds SF1,

FIG. 6D illustrates clearance of Mtb upon administration of test compounds SF1 and SF2 compared to untreated group (UT) in lungs.

FIG. 6E illustrates clearance of Mtb upon administration of test compounds SF2 (2×) and SF3 compared to untreated group (UT) in lungs.

FIG. 6F illustrates clearance of Mtb upon administration of test compounds SF1 and SF2 compared to untreated group (UT) in spleen.

FIG. 6E illustrates clearance of Mtb upon administration of test compounds SF2 (2×) and SF3 compared to untreated group (UT) in spleen.

FIG. 7 illustrates the pathway of de novo histidine biosynthesis.

FIG. 8A illustrates functional/biological unit of the enzyme IGPD.

FIG. 8B illustrates one active site composition of IGPD.

FIG. 9 illustrates in vitro cytotoxicity study against different cell lines.

FIG. 10A illustrates histopathology of the kidney when treated with SF2.

FIG. 10B illustrates histopathology of the kidney when treated with SF3.

FIG. 11A illustrates histopathology of the liver when treated with SF2.

FIG. 11B illustrates the histopathology of the liver when treated with SF3.

FIG. 12A illustrates effect of Compound SF2 in different doses after a single dose administration on the body weight gain pattern of female mice.

FIG. 12B illustrates effect of Compound SF3 in different doses after a single dose administration on the body weight gain pattern of female mice.

### DETAILED DESCRIPTION OF THE INVENTION

In the following description, reference is made to the accompanying drawings where, by way of illustration, specific embodiments of the invention are shown. It is to be understood that other embodiments may be used, and other changes may be made without departing from the scope of the present invention. Accordingly, the drawings and detailed description are to be regarded as illustrative in nature and not as restrictive.

As will be realized in the following description, the invention is capable of other and different embodiments and its several details are capable of modifications in various respects, all without departing from the scope of the present invention. This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### Definitions

The term “pharmaceutically effective amount” or “therapeutically effective amount” or “effective amount” as used herein refers to an amount, which has a therapeutic effect or is the amount required to produce a therapeutic effect in a subject. For example, a therapeutically or pharmaceutically effective amount of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). The pharmaceutically effective amount depends on several factors, including but not limited to, the microorganism (e.g. bacteria) involved, characteristics of the subject (for example height, weight, sex, age and medical history), severity of infection and the particular type of the antibacterial agent used. For prophylactic treatments, a therapeutically or prophylactically effective amount is that amount which would be effective in preventing a microbial (e.g. bacterial) infection.

The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention includes oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In case of a pharmaceutical composition comprising more than one ingredient (active or inert), one of way of administering such composition is by admixing the ingredients (e.g. in the form of a suitable unit dosage form such as tablet, capsule, solution, powder and a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g. bacteria). The term also includes maintenance of on-going metabolic processes of a microorganism, including processes that keep the microorganism alive.

The term, “effectiveness” as used herein refers to ability of a treatment or a composition or one or more pharmaceutically active ingredients to produce a desired biological effect in a subject. For example, the term “antibacterial effectiveness” of a composition or an antibacterial agent refers to the ability of the composition or the antibacterial agent to prevent or treat the microbial (e.g. bacterial) infection in a subject.

The term, “treatment” or “treating,” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

The term “antibacterial agent” as used herein refers to any substance, compound, composition of compounds or a combination of substances or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment.

Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins.

The term “pharmaceutically “excipient” refers to a compound or material used to facilitate administration of a compound, including for example, to increase the compound's solubility. Typical, non-limiting examples include Calcium carbonate, Lactose, anhydrous Lactose monohydrate, Mannitol, Magnesium carbonate, Magnesium oxide, Microcrystalline cellulose, Sorbitol, Starch, micro-silica gel and talcum powder and lubricant is selected from magnesium stearate, glyceryl monostearate, stearic acid, talc, DMSO, Tween 80, Normal saline 0.9% sodium chloride and the like.

The term “subject” herein refers to vertebrates or invertebrates, including a mammal. The term “subject” includes humans, canine, feline, bovine, ovine, caprine, porcine, avian, piscine, and equine species.

In one aspect, there is provided an antibacterial compound of Formula 1 or pharmaceutically acceptable salt thereof;

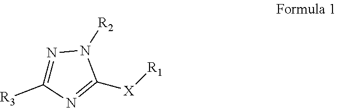

wherein X is CH, S, CH—NH₂;
R₁ is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

In one embodiment, the compound with Formula 1 is an imidazole glycerol phosphate dehydratase (IGPD) inhibitor.

In another embodiment, the compound with Formula 1 is selected from
1-(2-methyl-1,2,4-triazol-3-yl)ethanamine;
2-methyl-1-(2-methyl-1,2,4-triazol-3-yl)propan-1-amine;
2-(1H-1,2,4-triazol-5-ylsulfanyl)ethanamine;
N-[2-(1H-1,2,4-triazol-5-ylsulfanyl) ethyl]prop-2-yn-1-amine;
N-[(5-ethyl-2-methyl-1,2,4-triazol-3-yl) methyl]propan-2-amine;
5-(azetidin-3-ylsulfanyl)-1H-1,2,4-triazole;
(1-butyl-1,2,4-triazol-3-yl)methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl)methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine; and
N′-methyl-N′-propan-2-yl-N-[(2-propyl-1,2,4-triazol-3-yl) methyl]propane-1,3-diamine.

In another aspect is provides a compound of Formula 1 or pharmaceutically acceptable salt, solvate, or hydrate thereof as a growth inhibitor of Mycobacterium tuberculosis;

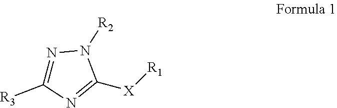

wherein X is CH, S, CH—NH₂;
R₁ is C1-C8 alkyl, substituted alkyl, alkyl amine, substituted amine; preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropylethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

In one embodiment, the compound with Formula 1 is an imidazole glycerol phosphate dehydratase (IGPD) inhibitor.

In another embodiment, the compound with Formula 1 is:
1-(2-methyl-1,2,4-triazol-3-yl)ethanamine;
2-methyl-1-(2-methyl-1,2,4-triazol-3-yl)propan-1-amine;
2-(1H-1,2,4-triazol-5-ylsulfanyl)ethanamine;
N-[2-(1H-1,2,4-triazol-5-ylsulfanyl)ethyl]prop-2-yn-1-amine;
N-[(5-etbyl-2-methyl-1,2,4-triazol-3-yl)methyl]propan-2-amine;
5-(azetidin-3-ylsulfanyl)-1H-1,2,4-triazole;
(1-butyl-1,2,4-triazol-3-yl)methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl)methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine;
N′-methyl-N′-propan-2-yl-N-[(2-propyl-1,2,4-triazol-3-yl)methyl]propane-1,3-diamine.

In a further aspect, is provided a compound of Formula 1 or pharmaceutically acceptable salt, solvate, or hydrate thereof as an imidazole glycerol phosphate dehydratase (IGPD) inhibitor for use in treatment or prevention or amelioration of tuberculosis.

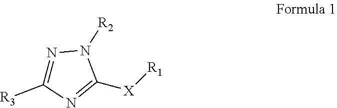

wherein X is CH, S, CH—NH₂;
R1 is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine,N1-ethyl-N1-propylethane-1,2-diamine, cyclobutyamine, phosphate, sulphate;
R2 is hydrogen, alkyl, substituted alkyl; preferably R2 is methyl, propyl, isopropyl;
R3 is hydrogen, alkyl, substituted alkyl; and preferably R3 is methyl, propyl, isopropyl.

In one embodiment, the compound with Formula 1 is an imidazole glycerol phosphate dehydratase (IGPD) inhibitor.

In another embodiment, the IC50 value for the compounds with Formula 1 is in the range of 33 to 77 μM, and the minimum inhibitor concentration is in the range of 31 to 500 μM.

In another embodiment, the compound with Formula 1 is selected from
1-(2-methyl-1,2,4-triazol-3-yl)ethanamine;
2-methyl-1-(2-methyl-1,2,4-triazol-3-yl)propan-1-amine;
2-(1H-1,2,4-triazol-5-ylsulfanyl)ethanamine;
N-[2-(1H-1,2,4-triazol-5-ylsulfanyl)ethyl]prop-2-yn-1-amine;
N-[(5-ethyl-2-methyl-1,2,4-triazol-3-yl)methyl]propan-2-amine;
5-(azetidin-3-ylsulfanyl)-1H-1,2,4-triazole;
(1-butyl-1,2,4-triazol-3-yl)methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl)methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine; and
N′-methyl-N′-propan-2-yl-N-[(2-propyl-1,2,4-triazol-3-yl)methyl]propane-1,3-diamine.

In a further aspect is provided a pharmaceutical composition comprising an effective amount of compound with Formula 1 or pharmaceutically acceptable salt thereof, together with at least one pharmaceutically acceptable carrier, diluent, excipient, and/or adjuvant.

In an embodiment, the effective amount of compound with Formula 1 is between 5 mg/kg to 5000 mg/kg; preferably 500 mg/kg.

In another embodiment, the excipients are selected from Calcium carbonate, Lactose, anhydrous Lactose monohydrate, Mannitol, Magnesium carbonate, Magnesium oxide, Microcrystalline cellulose, Sorbitol, Starch, micro-silica gel and talcum powder and lubricant is selected from magnesium stearate, glyceryl monostearate, stearic acid, talc, DMSO, Tween 80, Normal saline 0.9% sodium chloride and the like.

In another embodiment, the compound of Formula 1 is an imidazole glycerol phosphate dehydratase (IGPD) inhibitor.

In another embodiment, the compound with Formula 1 is selected from
1-(2-methyl-1,2,4-triazol-3-yl)ethanamine;
2-methyl-1-(2-methyl-1,2,4-triazol-3-yl)propan-1-amine;
2-(1H-1,2,4-triazol-5-ylsulfanyl)ethanamine;
N-[2-(1H-1,2,4-triazol-5-ylsulfanyl)ethyl]prop-2-yn-1-amine;
N-[(5-ethyl-2-methyl-1,2,4-triazol-3-yl)methyl]propan-2-amine;
5-(azetidin-3-ylsulfanyl)-1H-1,2,4-triazole;
(1-butyl-1,2,4-triazol-3-yl)methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl) methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine; and
N′-methyl-N′-propan-2-yl-N-[(2-propyl-1,2,4-triazol-3-yl)methyl]propane-1,3-diamine.

In a further aspect is provided a method for inhibiting Mycobacterium tuberculosis, wherein said method comprises administering a therapeutically effective amount of a compound with Formula 1 to a subject in need thereof.

In an embodiment, the subject is an animal selected from the group comprising human, canine, feline, bovine, ovine, caprine, porcine, avian, piscine, and equine species.

In another embodiment, the compound is administered to the subject in a dose in the range of 5 mg/kg to 5000 mg/kg body weight.

In another embodiment, the compound is administered to the subject by oral administration, parenteral administration or topical administration.

In another embodiment, the method further comprises administering one or more other antibacterial agents.

In another embodiment, the one or more other antibacterial agents are selected from rifampicin, isoniazid, pyrazinamide, amikacin, ethionamide, ethambutol, streptomycin, para-aminosalicylic acid, cycloserine, kanamycin, thioacetazone delamanid, moxifloxacin, gatifloxacin, ofloxacin, ciprofloxacin, sparfloxacin, clarithromycin, amoxycillin, rifamycins, rifabutin, rifapentine, or a combination thereof.

In another embodiment, the one or more other antibacterial agents and the compound with Formula 1 are administered simultaneously.

In another embodiment, the one or more other antibacterial agents and the compound of claim 1 are administered separately.

In another embodiment, the one or more other antibacterial agents and the compound with Formula 1 are administered sequentially.

In another aspect, is provided use of the compound of Formula 1 or pharmaceutically acceptable salts

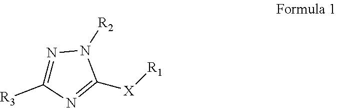

wherein X is CH, S, CH—NH₂;
R₁ is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl for preparation of medicament in treatment or prevention or amelioration of tuberculosis comprising administering a therapeutically effective amount of compound of Formula 1 or pharmaceutically acceptable salts thereof or pharmaceutical composition comprising a compound of Formula 1 or pharmaceutically acceptable salts thereof.

In an embodiment, the compound of Formula 1 is an imidazole glycerol phosphate dehydratase (IGPD) inhibitor.

In another embodiment, the effective amount of said medicament is between 5 to 2000 mg/kg, preferably 500 mg/kg.

In another aspect, the compound with Formula 1 include:

| Code | General 2D structure |
|---|---|
| SF14 | 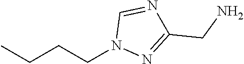 |
| SF18 | 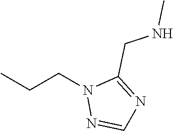 |
| SF5 | 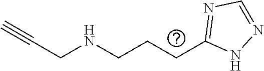 |
| SF3 | 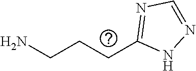 |
| SF44 | 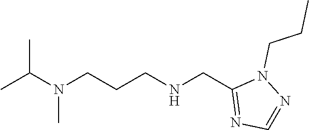 [SMILES: CCCn1ncnc1CNCCCN(C)C(C)C] |
| SF15 | 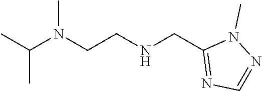 |
| SF34 | 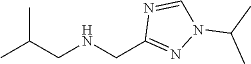 |
| SF1 | 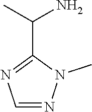 |
| SF36 | 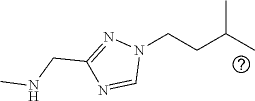 |
| SF37 | 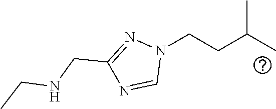 |
| SF43 | 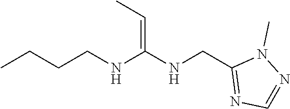 [SMILES: C/C=C(\NCCCC)NCc1ncnn1C] |
| SF42 | 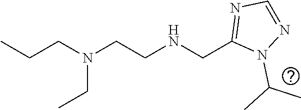 [SMILES: CCCN(CC)CCNCc1ncnn1C(C)C] |
| SF12 | 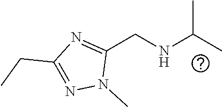 |
| SF2 | 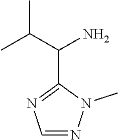 |
| SF13 | 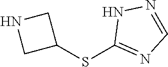 |

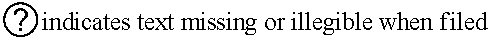

Herein, “comprising” means the term “comprising” and certain ingredients are defined as “consisting of” and “consisting essentially of”.

Herein “about” will be understood by persons of ordinary skill in the art and will vary to some extent on the context in which the term is used, “about” will mean up to plus or minus of the particular-term pharmaceutically acceptable ranges.

### WORKING EXAMPLES

The following examples are representative of the disclosure and provide detailed methods for preparing the compounds of the disclosure, including the preparation of the intermediate compounds. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g., by appropriately protecting interfering groups, by changing to other suitable reagents known in the art, or by making routine modifications of reaction conditions.

As used herein the symbols and conventions used in these processes, schemes, and examples, regardless of whether a particular abbreviation is specifically defined, are consistent with those used in the contemporary scientific literature, for example, the Journal of American Chemical Society.

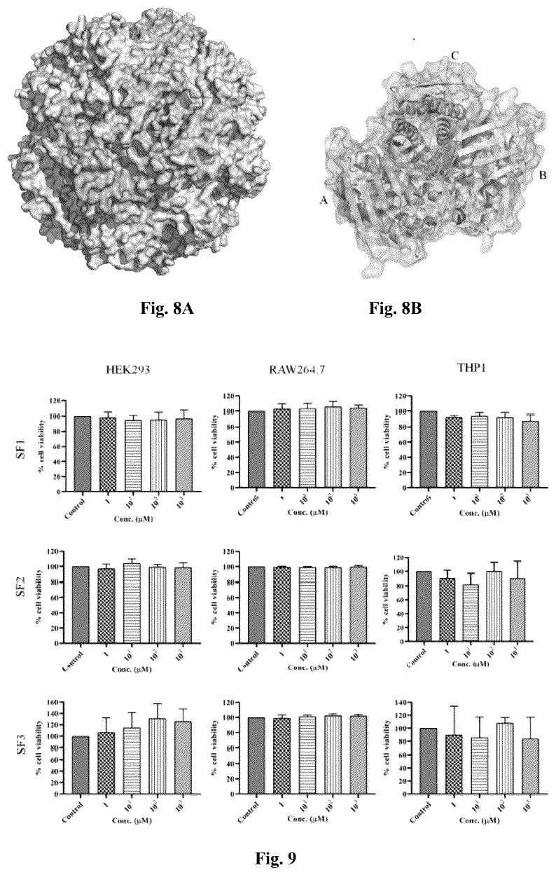

Over the last 25 years, plant IGPD, an enzyme that catalyzes the sixth step of the His pathway, has been an important herbicide target. The pathway has been provided in FIG. 7. A plethora of studies particularly in the area of developing potent herbicides targeting IGPD have led to the discovery of an ample number of small molecules. These studies showed that triazole and imidazole scaffold compounds are promising IGPD inhibitors. The availability of such a wealth of information provides a basis to examine whether triazole and imidazole scaffold inhibitors exhibit anti-TB potency by inhibiting the function of the His pathway of Mtb. Studies show that triazole and imidazole derivatives compounds have better prospects in inhibiting the function of IGPD. The functional unit of the enzyme IGPD is a 24-mer with 24 identical active sites (FIG. 8A. Each active site is asymmetric and made up with three subunits (FIG. 8B). By utilizing these structural information into the inhibitors design; a number of triazole derivative compounds (Table 1) and calculated the binding affinity of each of these compounds. The physicochemical properties together with the binding scores clearly suggest that these molecules have anti-TB potential and therefore warranted further investigation. In order to pursue further these molecules were studied in vitro and in vivo and investigated.

### Example 1: Screening of Compounds With Formula 1 on IGPD

IGPD, is an enzyme that catalyses the sixth step of the His pathway, has been an important herbicide target. The pathway has been provided in FIG. 7. The functional unit of the enzyme IGPD is a 24-mer with 24 identical active sites (FIG. 8A. Each active site is asymmetric and made up with three subunits (FIG. 8B). By utilizing this structural information into the inhibitors design; a number of triazole derivative compounds (Table 1) and calculated the binding affinity of each of these compounds. The physicochemical properties together with the binding scores clearly suggest that these molecules have anti-TB potential and therefore warranted further investigation.

One active site of IGPD consisting of three subunits was constructed and the active site area was chosen as a grid to dock each small molecule in this area. Features of small molecules, such as their structure and shape, play an important role in the interaction of residues present in the active site of the protein. This information could help build a specific and influential library of small molecules. Over the years, many compounds have been researched to identify drugs against TB, among which triazoles have been found to be the most explored and valuable scaffold. Triazole is a heterocyclic compound that includes a five-membered ring consisting of two carbon and three nitrogen atoms. The properties of triazole rings, such as hydrogen-binding ability, moderate dipole moment, and enhanced water solubility under in vivo conditions, are responsible for their enhanced biological activities. Ongoing research is being carried out across the globe to seek effective treatments for TB, which has suggested that a compound with a triazole motif is a promising inhibitor of IGPD. Using this as a basis, a library of compounds was created with triazole motifs.

A total of 50 compounds were shortlisted and set up for in silico virtual screening of the prepared IGPD receptor. In-house library compounds with Formula 1 are summarized in Table 1.

TABLE 1
The structures of the compounds as per Formula 1

| | |
|---|---|
| 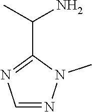 | SF1 |
| 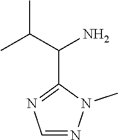 | SF2 |
| 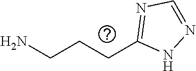 | SF3 |
| 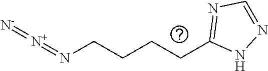 | SF4 |
| 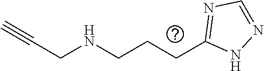 | SF5 |
| 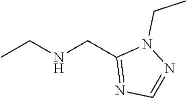 | SF6 |
| 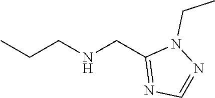 | SF7 |
| 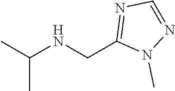 | SF8 |
| 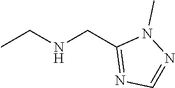 | SF9 |
| 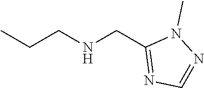 | SF10 |
| 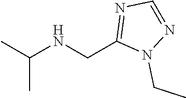 | SF11 |
| 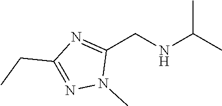 | SF12 |
| 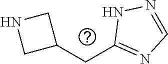 | SF13 |
| 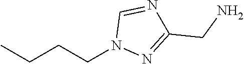 | SF14 |
| 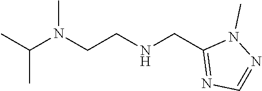 | SF15 |
| 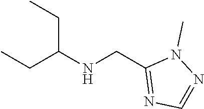 | SF16 |
| 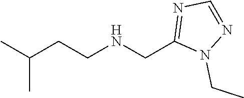 | SF17 |
| 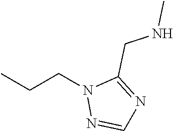 | SF18 |
| 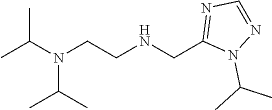 | SF19 |
| 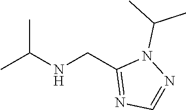 | SF20 |
| 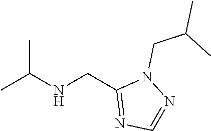 | SF21 |
| 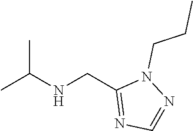 | SF22 |
| 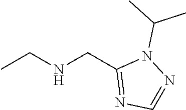 | SF23 |
| 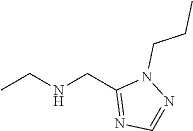 | SF24 |
| 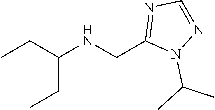 | SF25 |
| 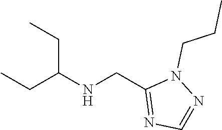 | SF26 |
| 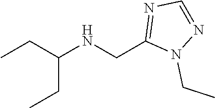 | SF27 |
| 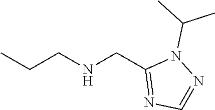 | SF28 |
| 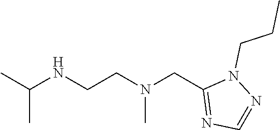 | SF29 |
| 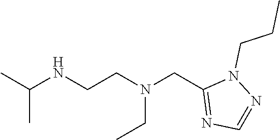 | SF30 |
| 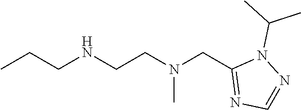 | SF31 |
| 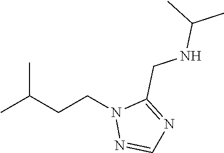 | SF32 |
| 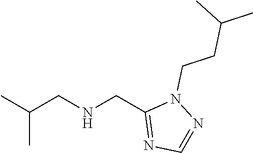 | SF33 |
| 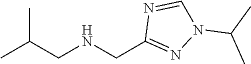 | SF34 |
| 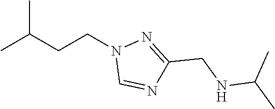 | SF35 |
| 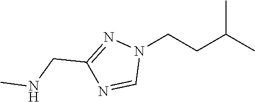 | SF36 |
| 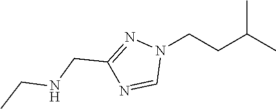 | SF37 |
| 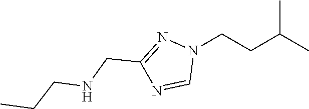 | SF38 |
| 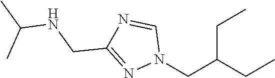 | SF39 |
| 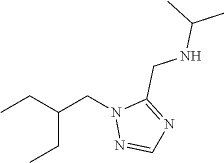 | SF40 |
| 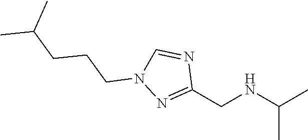 | SF41 |
| 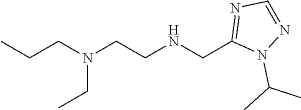 | SF42 |
| 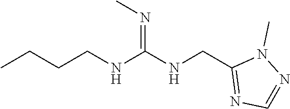 | SF43 |
| 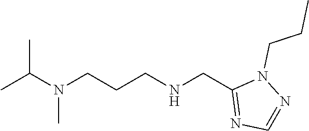 | SF44 |
| 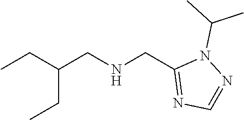 | SF45 |
| 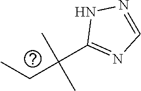 | SF46 |
| 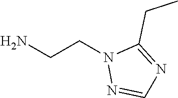 | SF47 |
| 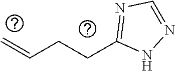 | SF48 |
| 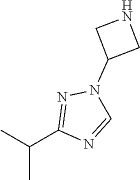 | SF49 |
| 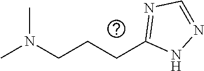 | SF50 |

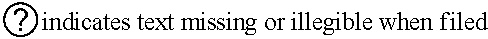

Each of these molecules was docked in this area using a guided docking protocol with Schrodinger drug discovery software. The docking score and binding energy are listed in the Table 2 of top 15 compounds.

TABLE 2
Docking scores and binding energies of top 15 compounds

| Code | Docking score (kcal/mol) | MMGBSA dG Bind (kcal/mol) |
|---|---|---|
| SF14 | −6.539 | −8.40 |
| SF18 | −5.701 | −18.38 |
| SF5 | −5.589 | −27.20 |
| SF3 | −5.587 | −29.59 |
| SF44 | −5.546 | −28.09 |
| SF15 | −5.317 | −20.41 |
| SF34 | −5.184 | −9.66 |
| SF1 | −4.936 | −11.97 |
| SF36 | −4.863 | −8.44 |
| SF37 | −4.713 | −34.68 |
| SF43 | −4.493 | 5.26 |
| SF42 | −4.435 | −26.53 |
| SF12 | −4.370 | −25.71 |
| SF2 | −4.368 | −11.65 |
| SF13 | −4.327 | −20.11 |

Compounds SF1, SF2 and SF3 was selected for further studies.

### Example 2: Physicochemical Characterization

The compounds with Formula 1 show appropriate gastrointestinal absorption and low penetration of the blood-brain barrier similar to that of one of the first-line TB drugs, isoniazid (INH). Qikprop analysis categorized these molecules as drug-like with zero violation of a widely regarded, “Lipinski rule of five” (Table 3A), and most of the properties of the compound were in the range of 95% known drugs.

TABLE 3A
Physicochemical properties of three Triazole-based compounds

| | Molecular | No. of | | | | | | | |
|---|---|---|---|---|---|---|---|---|---|
| | Weight | Rotatable | H-Bond | H- Bond | | | Dipole | Volume | |
| Compounds | (Da) | Bonds | Donors | Acceptors | PSA | Log P_{o/w} | Moment | (SASA) | CNS |
| SF1 | 126.16 | 1 | 2 | 3 | 56.73 | −0.70 | 5.501 | 328.12 | 1 |
| | (130-725) | (0-15) | (0-6) | (2-20) | (7-200) | (−2.0-6.5) | (1-12.5) | (300-1000) | (−2.0-2.0) |
| SF2 | 190.67 | 2 | 2 | 3 | 56.73 | 1.06 | 5.232 | 367.18 | 1 |
| SF3 | 217.12 | 3 | 3 | 3 | 92.89 | 1.49 | 4.203 | 340.84 | −1 |

* Values in parenthesis are for 95% of known drugs.

ProTox Cytotoxicity predictions suggested mild probabilities for compounds to be toxic or carcinogenic (Table 3B).

TABLE 3B
Toxicological predictions of the Compounds
B. Toxicological predictions of the Compounds

| | LD₅₀ | Toxicity | Toxicity endpoints | | | |
|---|---|---|---|---|---|---|
| Compounds | (mg/Kg) | class | Carcinogenicity | Immunotoxicity | Mutagenicity | Cytotoxicity |
| SF1 | 450 | IV | Active | Inactive | Active | Inactive |
| | | | (0.73) | (0.99) | (0.56) | (0.77) |
| SF2 | 2500 | V | Active | Inactive | Active | Inactive |
| | | | (0.61) | (0.99) | (0.59) | (0.74) |
| SF3 | 2330 | V | Active | Inactive | Active | Inactive |
| | | | (0.59) | (0.98) | (0.53) | (0.61) |

*Values in the parenthesis are the probability value (from 0 to 1 giving confidence of the prediction result).

Thus, illustrating that Triazole scaffold compounds show drug-like properties.

### Example 3: Enzyme Substrate Interactions Studies

### I. Over Expression and Purification of IGPD

The over-expression of the enzyme in a Mycobacterium smegmatis (Msmeg) over expression system and its purification to a high degree of homogeneity were carried out. For over expression, Rv1601-pYUB1062 expression vector was electroporated into competent Msmeg strain mc²4517 cells. A single transformed colony was revived in 10 ml of Luria Bertani (LB) broth supplemented with 0.05% Tween 80, 0.2% glycerol, and the antibiotics kanamycin (25 μg/ml) and hygromycin B (50 μg/ml). The culture was grown at 310 K, and 180 rpm for 24 h. 1 ml of the primary culture was inoculated into 50 ml of the same medium and grown at 310 K, 180 rpm until A600 reached 0.6-0.8. Subsequently, the culture was diluted 30-fold into 1.5 liters of the same medium to make a secondary culture and grown to mid-exponential phase (A600=0.8) at 310 K, 180 rpm and then induced with 0.02% acetamide. After 24 h of induction, cells were harvested by centrifugation at 10,000×g for 20 min. The cell pellet was resuspended in 50 ml of 20 mM Tris, pH 8.5, 200 mM NaCl, 5% glycerol, and 20 mM imidazole buffer with one Complete Mini, EDTA-free protease inhibitor tablet (Roche Applied Science). The cells were lysed at 277 K under high pressure (25,000 p.s.i.) using a cell disrupter (Constant Systems Ltd., UK). The lysate was centrifuged at 10,000×g for 45 min at 277 K to the remove unbroken cells and inclusion bodies. The supernatant was then loaded onto an equilibrated nickel-nitrilotriacetic acid affinity column. The column was washed with 20 mM Tris, pH 8.5, 200 mM NaCl, 5% glycerol, and 50 mM imidazole to remove non-specifically bound proteins. Subsequently, Rv1601 was eluted from the column with 300 mM imidazole in the same buffer. The eluted protein was concentrated and further purified by size-exclusion chromatography using a HiLoad 16/600 Superdex 200 prep grade column (GE Healthcare) in 20 mM Tris, pH 8.5, and 200 mM NaCl buffer. The degree of purity of the IGPD (Rv1601) was examined by 10% SDS-PAGE.

### II. Biochemical Assay

The activity of the enzyme was determined using a previously described stopped-assay protocol with minor modifications. Briefly, the reaction mixture consisted of 40 mM Triethanolamine (TEA) buffer pH 7.7, 50 mM MnCl₂, 25 mM β-Mercaptoethanol, and 2 μg (1.16 μM) of the enzyme in a reaction volume of 75 μl. Initially, enzyme and substrate concentrations were optimized by bringing the output signal to the reading capacity of the instrument. Further, enzyme concentration was optimized for a linear range of initial velocity up to 60 s, maintaining the same substrate concentration. For the IC50 study, the reactions were carried out at 310 K using the fixed concentration of IGP (0.166 mM) and the different concentrations of triazole scaffold-based inhibitors (0.010 to 2.0 mM) in a serial 2-fold manner. The reactions were stopped by adding 250 μl of 1.43 M sodium hydroxide. Different time points were noted for the kinetics followed a gradation of 30 s. The reaction mixture was then incubated at 318 K for 20 min to convert the product imidazole acetol phosphate (IAP) into an enolized form, whose absorbance was read at 280 nm in a Shimadzu UV spectrophotometer against an appropriate blank. The extinction coefficient of IAP formed under these conditions was 5310 M⁻¹ cm⁻¹, as reported previously and this value was used for the calculation of kinetic parameters. Data points were plotted using Graph Pad Prism 6.0.

### III. Characterization of the Kinetics Parameters

In order to examine the degree of affinity and specificity of each enzyme/inhibitor complex, in vitro kinetics analysis was carried out.

The antitubercular activity of the compound was tested by performing a Resazurin Assay (REMA). Briefly, Mtb H37Rv were grown to mid-logarithmic phase (A600=1-1.5) in Middlebrook 7H9 broth (Difco) supplemented with 10% OADC, 0.2% glycerol and 0.05% Tween 80 under aerobic conditions with shaking at 190 rpm. The culture was resuspended by passaging it 10-15 times through a 26.5-gauge needle and diluting it in growth medium to A600=0.001. All cultures were grown at 37° C. in a shaker incubator. Drug susceptibility testing using resazurin was performed under aerobic conditions similar to that described for Mtb with minor modifications. The assay was performed in clear-bottomed, 96-well microplates (NUNC-Microwell, Thermo scientific). An initial stock solution of inhibitor was prepared in sterile deionized water, and subsequent 2-fold serial dilutions were prepared in 0.1 ml of 7H9-S/Dubos-S medium supplemented with 0.2% glycerol (without Tween 80) in the 96-well microplates. Approximately 104 cells were added per well in a volume of 0.1 ml. Control wells containing [Mtb only and Medium+Compound (M+C)] were included in the plate setup. The plates were incubated at 37° C. for 5 days. After that, 20 μl of 0.02% resazurin and 12.5 μl of 20% Tween 80 were added. The wells were observed after 24 and 48 h for a color change from blue to pink. Visual minimum inhibitory concentration (MIC) was defined as the lowest concentration of drug that prevented a color change.

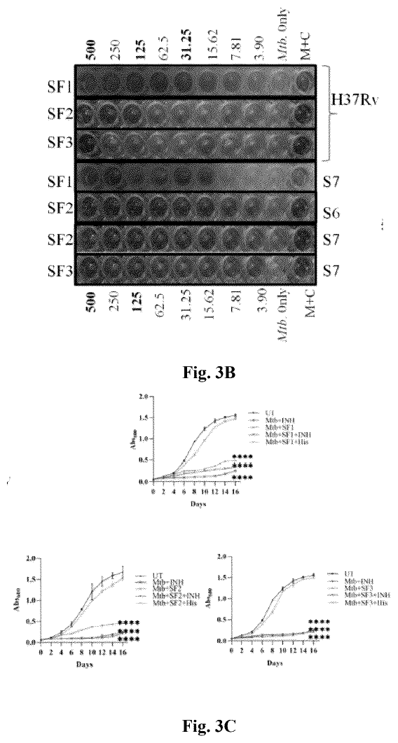

It was found that the IC₅₀ of inhibitors lie in the range of 33.46 μM (SF1), 44.09 μM (SF2), and 76.95 μM (SF3), calculated by plotting log (inhibitor concentration) vs response, four-parameter variable slope curve (FIG. 3A). These compounds inhibit the growth of H37Rv as well as drug resistant clinical isolates (S6-Resistant to INH+Rifampicin & S7-Resistant to INH) in minimal media with MIC values in micromolar range as confirmed by alamarBlue assay (Table 5, and FIG. 3B).

TABLE 4
Potency (MIC99 values) of compounds against
H37Rv and drug resistant clinical isolates.

| Compounds | MIC₉₉ (H37Rv) μM | MIC₉₉ (S6) μM | MIC₉₉ (S7) μM |
|---|---|---|---|
| SF1 | 31.25 | — | 250 |
| SF2 | 125 | 125 | 125 |
| SF3 | 500 | — | 250 |

Further, the effect on the growth of Mtb inhibited by these compounds following histidine supplementation in minimal media. As expected, histidine supplementation nullifies the inhibitory effects of the compounds and restores the Mtb growth (FIG. 3C), confirming that these compounds indeed target the histidine biosynthesis.

### IV. X-Ray Crystal Structures of Enzyme/Inhibitor Complexes

Elucidating enzyme/inhibitor interactions greatly assists in understanding the mechanism of drug action and helps in improving molecular design to achieve higher potency. In this regard, the crystal structures of IGPD-inhibitor complexes were individually determined.

To map the enzyme/inhibitor interactions, X-ray studies of enzyme/inhibitor complex were carried out. For this, IGPD crystals were soaked in 0.5 mM of inhibitor solution for 10 min and subsequently diffraction data were collected and processed. All the reported IGPD/inhibitor complex structures were refined in a similar manner. To begin with, each structure was subjected to 50 cycle of rigid body refinement followed by 100 cycles of maximum-likely-hood positional refinements The electron density maps for both 2|Fo|−|Fc| (at 1σ level) and |Fo|−|Fc| (at 3σ) were examined for the entire poly peptide chain and the model was corrected wherever necessary and subsequently water molecules were included into the model and positional and B-factor refinement were carried out.

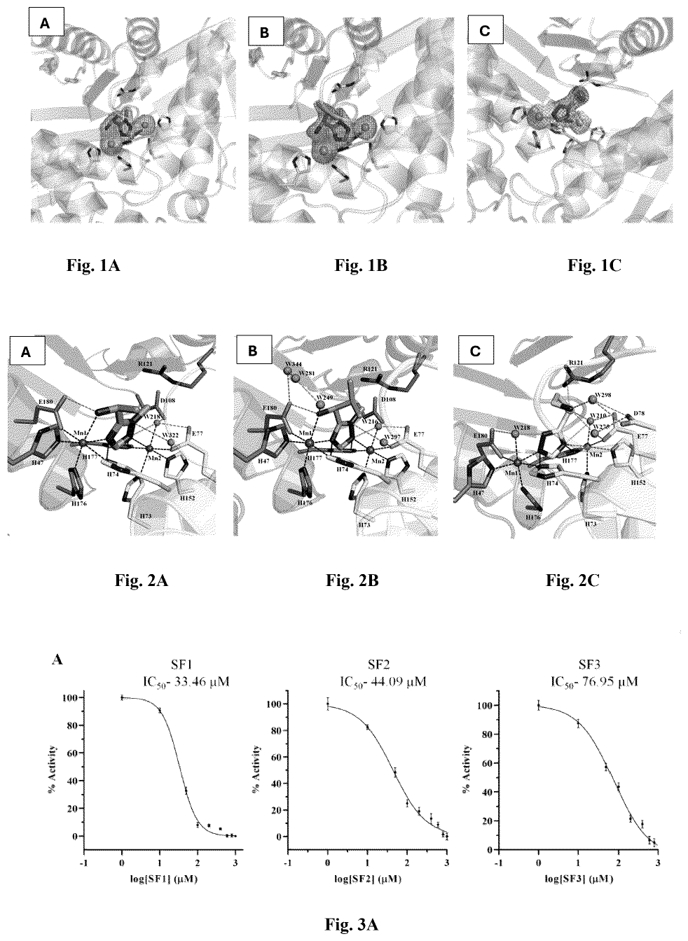

Examination of the 2|Fo|−|Fc| (at 1σ level) and |Fo|−|Fc| (at 3σ) confirmed the binding at the enzyme's active site. Each inhibitor was modeled into the electron density and subsequently 100 cycles of positional refinement was carried out. The stereo chemical acceptability of all three enzyme/inhibitor complex structures were checked using Ramachandran plot analysis. FIG. 1A illustrates Electron density maps of the X-ray crystal structures of enzyme/inhibitor complex SF1. FIG. 1B illustrates Electron density maps of the X-ray crystal structures of enzyme/inhibitor complex SF2, and FIG. 1C illustrates Electron density maps of the X-ray crystal structures of enzyme/inhibitor complex SF3. FIGS. 1A-1C shows the non-protein electron density peaks, particularly in the active site area, showing the presence of significant 2|Fₒ|−|F_{c}| and |Fₒ|−|F_{c}| electron density peaks resembling the shape of these inhibitors, demonstrating the inhibitors' binding at the active site of the enzyme. The critical requirements for the binding at the active site are a ring which allows the formation of coordination bonds to the Mn²⁺ ions and a negatively charged group is separated by 2-3 carbon chain from the ring. The difference in Fourier map |Fo|−|Fc| (is shown in green grids) at 3σ contour level for all three inhibitors in FIG. 1A, 1B, and 1C suggest that these inhibitors bind at the active site pocket of the enzyme. FIG. 2A illustrates interactions of IGPD with SF1 inhibitors. FIG. 2B illustrates interactions of IGPD with SF2 inhibitor, and FIG. 2C illustrates interactions of IGPD with SF3 inhibitor. From FIGS. 2A-2C illustrates the triazole ring of each inhibitor is anchored between these two active site manganese ions. The binding mode of the triazole ring is same for all inhibitors. The two active site manganese atoms are coordinated with the two nitrogen atoms of the triazole ring. The active site is made up of mainly histidine-rich amino acids. The amino acid residues involved in the interactions are shown in stick representation, and two Mn2⁺ are shown in spheres with the Purple-blue color in FIG. 2A-2C.

The data collection, data processing and refinement statistics are presented in the Table 5.

TABLE 5

| | SF1 | SF2 | SF3 |
|---|---|---|---|
| Space group | P432 | P432 | P432 |
| Unit cell | [symbol] | [symbol] | [symbol] |
| Dimensions ( [symbol] ) | [symbol] | [symbol] | [symbol] |
| Temperature (K) | 100 | 100 | 100 |
| Resolution ( [symbol] ) | [symbol] | [symbol] | [symbol] |
| | [symbol] | [symbol] | [symbol] |
| Unique reflections | [symbol] | [symbol] | [symbol] |
| [symbol] | [symbol] | [symbol] | [symbol] |
| Completeness (%) | [symbol] | [symbol] | [symbol] |
| Redundancy | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

| | | | |
|---|---|---|---|
| Protein | [symbol] | [symbol] | 30.75 |
| Ligand | 36.36 | 42.74 | [symbol] |
| Metal Ions | 22.15 | [symbol] | 26.53 |
| Solvent | 33.13 | 37.71 | 32.62 |

[symbol]

| | | | |
|---|---|---|---|
| Bond length ( [symbol] ) | 0.014 | 0.016 | 0.013 |
| Bond angle ( [symbol] ) | 1.91 | 2.09 | [symbol] |

[symbol]

| | | | |
|---|---|---|---|
| Preferred regions | [symbol] | 94.15 | [symbol] |
| Allowed regions | 4.26 | 5.32 | [symbol] |
| Disallowed regions | [symbol] | 0.53 | 0.53 |
| PDB ID | 7DDV | 7FCY | 7DNQ |

[symbol]

[symbol]

indicates data missing or illegible when filed

### Example 4. Triazole Compounds Clear Mtb Infection in Macrophages

To examine the in vitro potency of these compounds, macrophages cell lines (RAW 264.7) were infected with Mtb at Multiplicity of Index (MOI)=10 and observed the bacterial load in the presence of various concentrations of each compound.

H37Rv bacterial strain was grown in Middlebrook 7H9 broth (Difco) supplemented with 10% OADC, 0.2% glycerol and 0.05% Tween-80 until the mid-log phase. The bacteria were harvested, washed with RPMI 1640 and re-suspended in the same media (Antibiotic Free). Bacteria were quantified by measuring the absorbance at a wavelength 600 nm. The Raw cells 264.7 (macrophages) were seeded in 6 well plates at 106 cells/well and incubated for 3 h at 37° C. Macrophage monolayer was then washed with PBST and incubated with 2 ml infection media (RPMI 1640 medium supplemented with 10% FBS) containing 1×10⁷ cells of Mtb H37Rv (MOI=10) at 37° C. under 5% CO₂ for 3 h. Then the cells were washed twice with warm PBST to remove extracellular bacteria and supplemented with RPMI (without antibiotics) and a range of 0, 15, 30, 60, 120, 240 μM of Test compounds were added in respective wells. After 72 h of infection, media was aspirated from infected macrophage wells, and 500 μl of ice-cold sterile lysis solution (0.05% SDS, w/v in H2O) was added to each well. The lysates were further centrifuged at 1500 rpm for 10 min and transferred to new tube and serial dilutions were prepared. These dilutions were plated on 7H10 agar plates supplemented with OADC. The plates were kept in a CO₂ incubator for 3-4 weeks and subsequently the colony forming units (CFU) were counted.

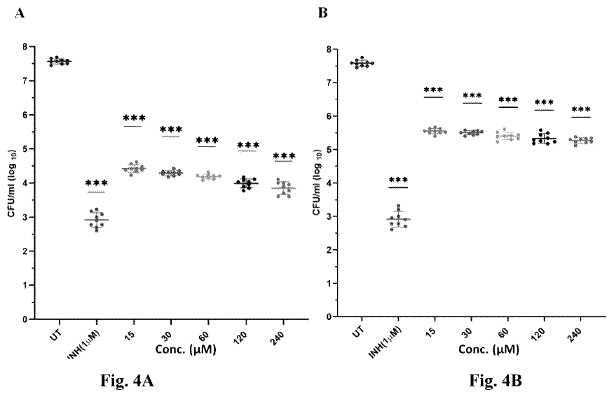

FIG. 4 illustrates the efficacy testing in Mtb infected macrophage model. As shown in FIG. 4, bacterial load is reduced significantly after 3 days of treatment, showing approximately a thousand-fold decline for SF1 (FIG. 4A) and hundred-fold decline for SF2 treatment (FIG. 4B). Although, SF3 compound inhibited the IGPD activity and curbed Mtb growth in culture medium but did not reduce the bacterial load in Mtb infected-macrophages at concentrations up to 1× of MIC.

### Example 5. Growth Kinetics of Mtb Strains

The Mtb strain H37Rv was revived from glycerol stock and/or single colony picked from 7H10 agar medium and inoculated into 10 ml of 7H9 broth medium. Upon reaching mid-logarithmic phase (A600˜0.8), the cultures were diluted again to A600˜0.05. Cultures were then challenged with 1×MIC of inhibitors. Cultures were maintained for 21 days at 37° C. and 100 r.p.m. A600 was determined at specific intervals. The growth kinetics was analyzed for three different compounds in three independently grown bacterial cultures (n=3).

### I. Cytotoxicity Assay

### Cell Culture Maintenance

RAW 264.7, HEK293 and THP-1 human monocytic cell line was obtained from ATCC. Cells were cultured in RPMI medium supplemented with 10% of heat-inactivated fetal bovine serum, 100 μM of penicillin and streptomycin, 50 μM neomycin and 200 mM of Glutamine in incubator at 37° C. under 5% CO₂ atmosphere. They were passaged every 2-3 days to prevent the cell density from exceeding one million per ml. Mycoplasma contamination was not found in any of the cells tested. For each set of experiments, THP-1 cells were differentiated into macrophages by exposure to 10 ng/ml (16 nM) of PMA in a plate appropriate to the test for 24 h, while PMA induced activation is not required for RAW 264.7 and HEK 293 cells. The density of 10⁴ cells/ml was used for all tests.

### II. In Vitro Assay

Cells which were in the log phase of growth were harvested and determined by cell count. 10⁴ cells/well were used for the assay (suggested cell density). The optimum cell density may vary between cell types. Cells were seeded in 96 well plates and exposed to test compounds for 72 h to determine the cytotoxic effect of test compounds on cell growth. After 72 h alamarBlue was added at an amount of 10% of the total media volume in the well. Plates were incubated with alamarBlue for 4-8 hr. The optimum incubation time varied between cell types. The viability of the cells was measured after 72 h incubation at 37° C., under 5% CO₂, using the alamarBlue assay by measuring the absorbance at different wavelengths of 570 nm and 600 nm after the required incubation. All Cytotoxicity experiments were performed in biological duplicates.

### Evaluation of In Vitro and In Vivo Toxicity of These Compounds

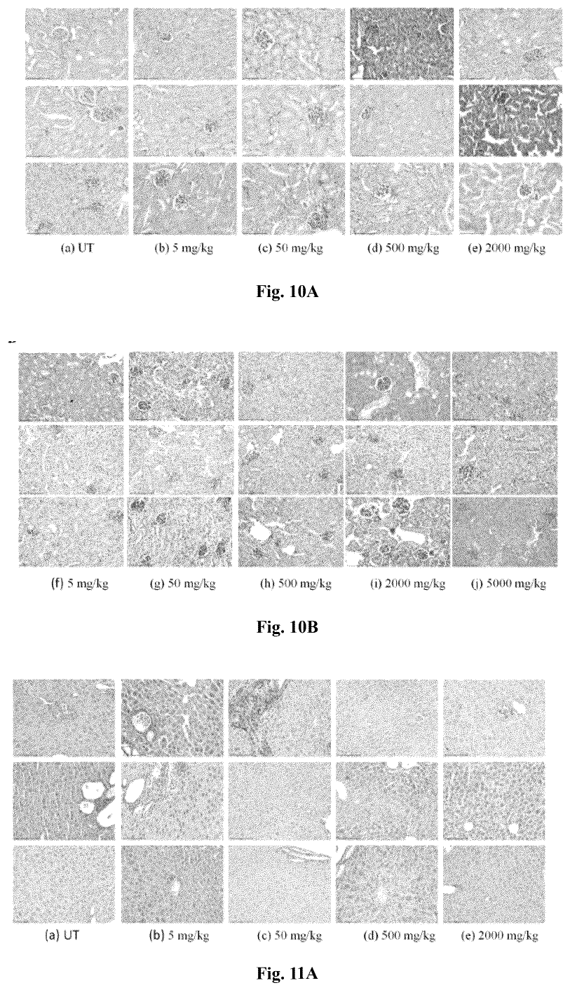

The water-soluble properties of these inhibitors make them better prospects for absorption. The toxicity of the said compounds were tested for both in vitro and in vivo systems. Using alamar Blue assays in Raw 264.7, THP1 and HEK-293 cell lines; it was observed that these compounds are non-toxic even at millimolar range concentrations (refer to FIG. 9). Further mice treated with different doses of each of these compounds and it was observed that all mice were alive after an oral administration of a single dose of 5, 50, 500, and 2000 mg/kg body weight of SF2 compound, while 2 mice of 2000 mg/kg body weight and 3 mice of 5000 mg/kg body weight died after administration of SF3 compound within four days. This suggested that the LD₅₀ value for SF2 is greater than 2000 mg/kg body weight and that of SF3 is below 5000 mg/kg body weight. All surviving mice behaved normally with no clinical signs of adverse effects/toxicity caused by the compounds after oral administration of the dose of 5, 50, 500 and 2000 mg/kg body weight of SF2 and SF3 during the entire 14 days acute oral toxicity study. Clinical signs such as slowness and weight decline appeared after 12-24 h in mice, after administration of single dose of 5000 mg/kg of SF3. After single dose of oral administration, mice body weight generally increased for all dose groups. FIG. 12A illustrates the effect of Compound SF2 at varying concentrations (5, 50, 500, 2000 mg/kg body weight) after a single dose administration on the body weight gain pattern of female mice monitored for 14 days and FIG. 12B illustrates the effect of Compound SF3 at varying concentrations (5, 50, 500, 2000, and 5000 mg/kg body weight) after a single dose administration on the body weight gain pattern of female mice monitored for 14 days. Data are shown as mean±s.d (n=5 for SF2 and SF3).

It can be seen that compared to the untreated group; body weight of mice administered with SF2 increased for all doses while a slight dip in weight for 5 mg/kg body weight on 7th day was observed. For SF3 treatments, body weight decreased for 5, 500, and 2000 mg/kg body weight and increased for 5000 mg/kg body weight compared to the untreated group was seen.

### III. Triazole Scaffold Compound Show Non-Toxic Effects in Mouse Model

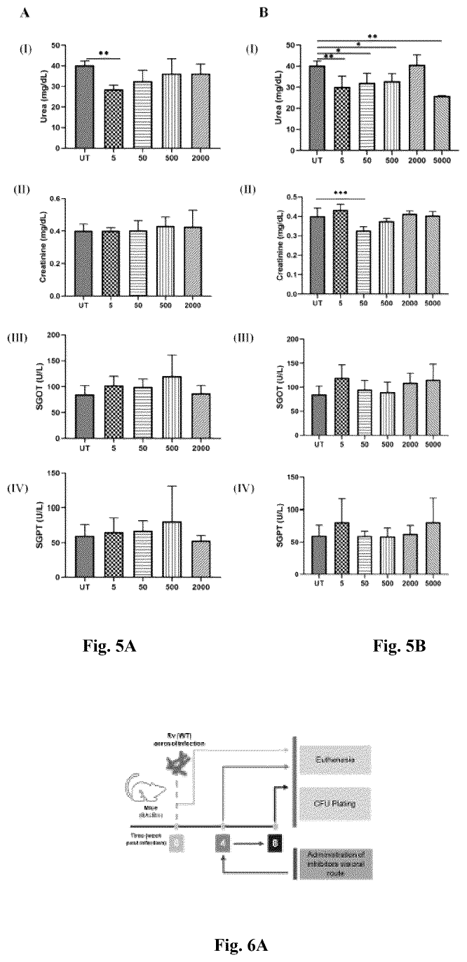

All the biochemical analysis was done using semi-automated machine, Coralyzer. After performing the serum analysis of SF2 and SF3 treatments for all doses, no significant changes were observed in the levels of Serum Glutamic Oxaloacetic Transaminase (SGOT), Serum Glutamic Pyruvic Transaminase (SGPT), creatinine, and urea, compared to the untreated group. As all values for the hepatic function marker enzymes (SGPT, SGOT) and renal and hepatic function marker enzymes (urea, creatinine) were in the normal range, it indicated no toxicity to liver and kidney (FIGS. 5A and 5B). FIG. 5A illustrates the effect in varying doses of SF2, while FIG. 5B illustrates the effect in varying doses of SF3.

Further, the histopathological analysis was performed to look for the tissue toxicity caused by the compounds used in the study. No significant changes were observed in the kidney and liver sections for the compounds SF2 and SF3.

FIG. 10A illustrates the effect of SF2 on kidney histology. Untreated and treated groups found to be normal with no pathological changes (b), (c), (d), and (e) indicates normal renal cortex, no leukocyte infiltration, no signs of edema, absence of necrotic foci, and normal tubular morphology at a dose of up to 2000 mg/kg body weight.

FIG. 10B illustrates the effect of SF3 on kidney histology. Untreated and treated groups found to be normal with no pathological changes (b), (c), (d), and (e) indicates normal renal cortex, no leukocyte infiltration, no signs of edema, absence of necrotic foci, and normal tubular morphology at a dose of up to 5000 mg/kg body weight.

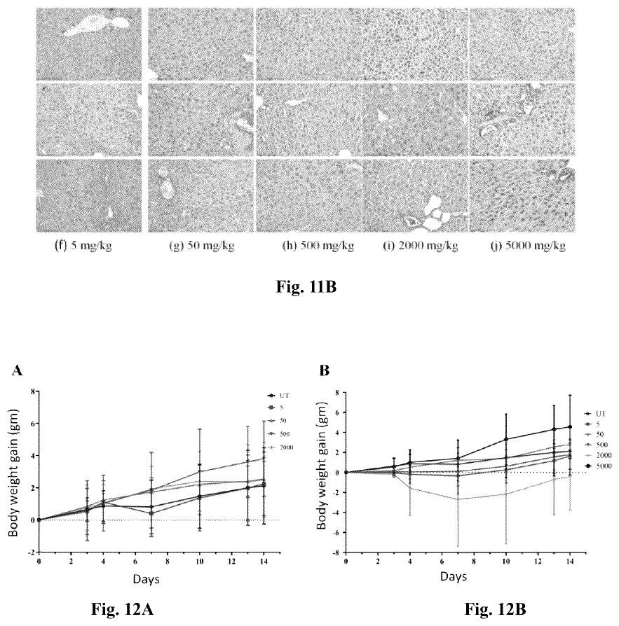

FIG. 11A illustrates the effect of SF2 on kidney histology. Untreated and treated groups found to be normal with no pathological changes (b), (c), (d), and (e) indicates normal Kupffer cells and no signs of granuloma, necrosis or infiltrations of degenerative cells at a dose of up to 2000 mg/kg body weight.

FIG. 11B illustrates the effect of SF3 on kidney histology. Untreated and treated groups found to be normal with no pathological changes (b), (c), (d), and (e) indicates normal Kupffer cells and no signs of granuloma, necrosis or infiltrations of degenerative cells at a dose of up to 5000 mg/kg body weight.

The histopathological parameters of liver and kidney in treated and the untreated groups were observed and confirmed by H&E staining.

### IV. Acute Toxicity Study

Healthy CD-1 female mice with weight 34±4 grams were used. The animals were housed individually in cages and maintained in a standard laboratory environment. Experimental protocols for the animal experiments were approved by the Institutional Animal Ethics Committee (IAEC) (IAEC#611/22) upon the recommendations and standards prescribed by the Committee for the Purpose of Control and Supervision of Experiments on Animals (CPCSEA), Government of India. Acute toxicity study was performed as per the up-and-down-procedure of Organization for Economic Cooperation and Development (OECD) guidelines 425. A limit dose of 2000 mg/kg body weight of compound was used involving three mice. Each mouse was treated with a single oral dose of 5, 50, 500, 2000 mg/kg body weight on Day 0. Animals were observed individually at least once during the first 30 min after dosing, periodically during the first 24 h and daily thereafter, for a total of 14 days for any clinical signs of toxicity or mortality. At the end of 14 day's observation period, the animals were anaesthetized, and their blood samples were collected through retro orbital bleeding for biochemical and histopathological studies, respectively. For biochemical analysis, blood was centrifuged at 4000 rpm at 4° C. for 10 min, serum was separated and Serum Glutamate Pyruvic Transaminase (SGPT), Serum Glutamate Oxaloacetic Transaminase (SGOT), creatinine, and, urea were estimated using a semi-automated Biochemical coralyzer. For histopathological studies, the animals were euthanized and the livers and kidneys were harvested and examined macroscopically. These organs were then preserved in 10% formalin made in PBS for histopathological examinations by standard techniques.

Analysis of the Acute Oral Toxicity Test of SF2 and SF3 on CD1 mice in 14 Days Study. Doses (mg/kg Body Weight) and Number of Deaths (D).

A.

| Compound | Doses (mg/kg) | Mice number initial | Mice number final | Total Deaths (D) |
|---|---|---|---|---|
| SF2 | 5 | 5 | 5 | 0 |
| | 50 | 5 | 5 | 0 |
| | 500 | 5 | 5 | 0 |
| | 2000 | 5 | 5 | 0 |

B.

| Compound | Doses (mg/kg) | Mice number initial | Mice number final | Total Deaths (D) |
|---|---|---|---|---|
| SF3 | 5 | 5 | 5 | 0 |
| | 50 | 5 | 5 | 0 |
| | 500 | 5 | 5 | 0 |
| | 2000 | 5 | 3 | 2 |
| | 5000 | 5 | 2 | 3 |

Sample preparation for H&E Staining experiments

For this, the liver and kidney tissue samples were embedded into wax blocks. The wax blocks were subjected to microtome to generate sections, which were then stained with Hematoxylin and Eosin dyes at a commercial facility. The tissue sections were viewed in a bright field microscope at 40× magnification.

### Animal Infection Study

Animal experiments were performed with approval from Institutional Animal Ethics Committee (IAEC Approval No: IAEC/THSTI/196) as per guidelines laid by CPCSEA, Government of India. Briefly, bacterial suspensions of wild type Mtb H37Rv at a density of 108 cells/ml was prepared in normal saline and female BALB/c (n=5 per group) were infected via aerosol route using Inhalation Exposure System (Glas-Col). Deposition of the bacteria as baseline infection was assessed by CFU at Day 1 post infection. Infection was allowed to progress through week 4 post infection at which animal groups (n=5) were administered orally with test compounds (mg/kg of animal body weight)−SF1 (100 mg/kg), SF2 (100 mg/kg), SF2 2× (200 mg/kg), SF3 (100 mg/kg), INH (10 mg/kg) and L-Histidine (100 mg/kg) for 5 days/week. Animals were euthanized at indicated time points post treatment, organs (lungs and/or spleens) were harvested and bacterial burden were determined by homogenization, serial dilution and CFU plating.

### Testing the Efficacies of These Compounds in Mouse Model of TB Infection

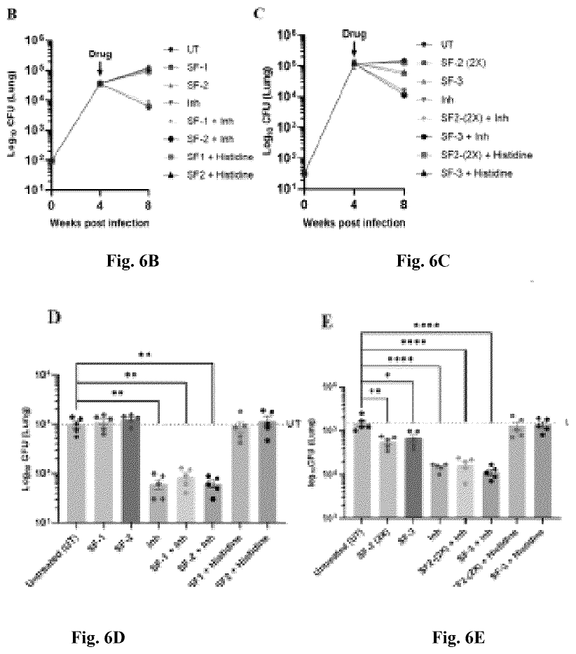

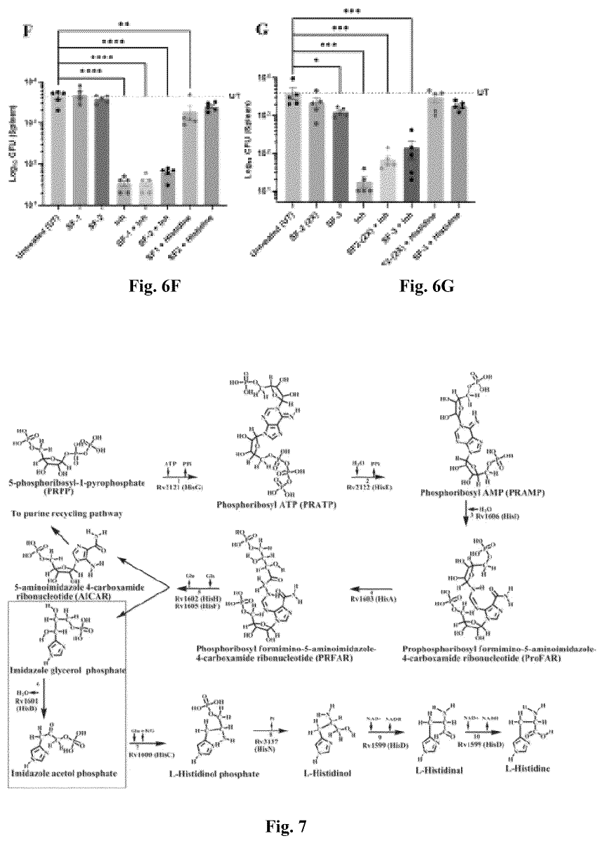

Based on absorption, cytotoxicity and in vitro macrophage clearance data, these inhibitor molecules were further tested for their efficacy against Mtb in animal model of TB infection. Briefly, Mtb infected BALB/c mice were administered with test compounds SF1 (100 mg/kg body weight), SF2 (100 mg/kg body weight), SF2 (2×) (200 mg/kg body weight) and SF3 (100 mg/kg body weight) 4 weeks post-infection (FIG. 6A). Eight weeks post-infection the mice were euthanized and the bacterial burden of the infected lungs and spleen were estimated by performing a colony forming unit assay (FIGS. 6B and C). It was observed that in comparison to the untreated control group, the lungs and spleen of mice treated with test compounds SF1 and SF2 showed no reduction in the bacterial burden (FIGS. 6D and F). However, compound SF3 at the same concentration demonstrated significant lowering of the bacterial burden in both lungs and spleen relative to the untreated (UT) mice (FIGS. 6E and G). Further, the SF2 compound at twice the concentration and observed that in comparison to the UT control group; there was a significant decrease in the bacterial load in the lungs of the mice (FIG. 6E). However, the reduction in the bacterial load in the infected spleen was not found to be statistically significant (FIG. 6G). These above findings indicate that the compound-mediated histidine limitation compromised bacterial survival inside the host microenvironment. Interestingly, supplementation of histidine in mice treated with SF2 (2×) and SF3 restored the bacterial burden to the untreated levels further validating the fact that the decrease in the bacterial load observed in the treated mice was a result of reduced biosynthesis of histidine.

1. An antibacterial compound of Formula 1 or pharmaceutically acceptable salt thereof: 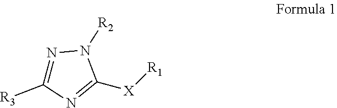
wherein X is CH, S, CH—NH₂;
R₁ is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine, N1-ethyl-N1-propylethane-1,2-diamine, cyclobutyamine, phosphate, sulphate;
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

2. The compound as claimed in claim 1, wherein the compound with Formula 1 is an imidazole glycerol phosphate dehydratase (IGPD) inhibitor.

3. The compound as claimed in claim 1, wherein the compound is selected from
1-(2-methyl-1,2,4-triazol-3-yl) ethanamine;
2-methyl-1-(2-methyl-1,2,4-triazol-3-yl)propan-1-amine;
2-(1H-1,2,4-triazol-5-ylsulfanyl)ethanamine;
N-[2-(1H-1,2,4-triazol-5-ylsulfanyl)ethyl]prop-2-yn-1-amine;
N-[(5-ethyl-2-methyl-1,2,4-triazol-3-yl)methyl]propan-2-amine;
5-(azetidin-3-ylsulfanyl)-1H-1,2,4-triazole;
(1-butyl-1,2,4-triazol-3-yl)methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl)methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine; and
N′-methyl-N′-propan-2-yl-N-[(2-propyl-1,2,4-triazol-3-yl)methyl]propane-1,3-diamine.

4. A compound of Formula 1 or pharmaceutically acceptable salt, solvate, or hydrate thereof as claimed in claim 1 for use a growth inhibitor of Mycobacterium tuberculosis, wherein said Formula 1 is: 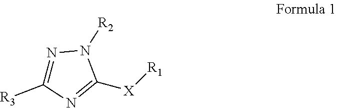
wherein X is CH, S, CH—NH₂;
R₁ is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropylethane-1,3-diamine, 1-butyl-2-methylguanidine, N1-ethyl-N1-propylethane-1,2-diamine, cyclobutyamine, phosphate, sulphate
R₂ is hydrogen, alkyl, substituted alkyl; preferably R₂ is methyl, propyl, isopropyl;
R₃ is hydrogen, alkyl, substituted alkyl; and preferably R₃ is methyl, propyl, isopropyl.

5. The compound as claimed in claim 4, wherein the compound is selected from
1-(2-methyl-1,2,4-triazol-3-yl)ethanamine;
2-methyl-1-(2-methyl-1,2,4-triazol-3-yl) propan-1-amine;
2-(1H-1,2,4-triazol-5-ylsulfanyl)ethanamine;
N-[2-(1H-1,2,4-triazol-5-ylsulfanyl)ethyl]prop-2-yn-1-amine;
N-[(5-ethyl-2-methyl-1,2,4-triazol-3-yl)methyl]propan-2-amine;
5-(azetidin-3-ylsulfanyl)-1H-1,2,4-triazole;
(1-butyl-1,2,4-triazol-3-yl) methanamine;
N′-methyl-N-[(2-methyl-1,2,4-triazol-3-yl)methyl]-N′-propan-2-ylethane-1,2-diamine;
N-methyl-1-(2-propyl-1,2,4-triazol-3-yl)methanamine;
2-methyl-N-[(1-propan-2-yl-1,2,4-triazol-3-yl)methyl]propan-1-amine;
N-methyl-1-[1-(3-methylbutyl)-1,2,4-triazol-3-yl]methanamine;
N-[[1-(3-methylbutyl)-1,2,4-triazol-3-yl]methyl]ethanamine;
N′-ethyl-N-[(2-propan-2-yl-1,2,4-triazol-3-yl)methyl]-N′-propylethane-1,2-diamine;
1-butyl-2-methyl-3-[(2-methyl-1,2,4-triazol-3-yl)methyl]guanidine; and
N′-methyl-N′-propan-2-yl-N-[(2-propyl-1,2,4-triazol-3-yl)methyl]propane-1,3-diamine.

6. A compound of Formula 1 or pharmaceutically acceptable salt, solvate, or hydrate thereof as claimed in claim 1 for use in treatment or prevention or amelioration of tuberculosis, wherein the Formula 1 is: 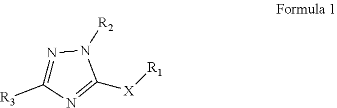
wherein X is CH, S, CH—NH₂;
R1 is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine,N1-ethyl-N1-propylethane-1,2-diamine, cyclobutyamine, phosphate, sulphate;
R2 is hydrogen, alkyl, substituted alkyl; preferably R2 is methyl, propyl, isopropyl;
R3 is hydrogen, alkyl, substituted alkyl; and preferably R3 is methyl, propyl, isopropyl.

7. The compound as claimed in claim 6, wherein the IC50 value for the compounds with Formula 1 is in the range of 33 to 77 μM, and the minimum inhibitor concentration is in the range of 31 to 500 μM.

8. A pharmaceutical composition comprising an effective amount of compound with Formula 1 or pharmaceutically acceptable salt thereof, together with at least one pharmaceutically acceptable carrier, diluent, excipient, and/or adjuvant, wherein the compound of Formula 1 is: 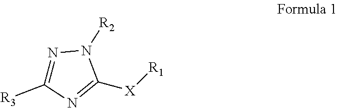
and wherein X is CH, S, CH—NH₂;
R1 is C1-C8 alkyl, substituted alkyl, alkyl amine, substituted amine;
preferably R1 is methyl, ethyl, propyl, butyl, pentyl, isopropyl, isobutyl, methyl amine, ethylamine, propylamine, isopropyl amine, isobutylamine, N-ethylprop-2-yn-1-amine, N-isopropyl propane-1,3-diamine, N1-isopropyl ethane-1,3-diamine, 1-butyl-2-methylguanidine,N1-ethyl-N1-propylethane-1,2-diamine, cyclobutyamine, phosphate, sulphate;
R2 is hydrogen, alkyl, substituted alkyl; preferably R2 is methyl, propyl, isopropyl;
R3 is hydrogen, alkyl, substituted alkyl; and preferably R3 is methyl, propyl, isopropyl.

9. The composition as claimed in claim 8, wherein the effective amount of compound with Formula 1 is between 5 mg/kg to 5000 mg/kg, preferably 500 mg/kg.

10. The composition as claimed in claim 8, wherein the excipients are selected from group comprising Calcium carbonate, Lactose, anhydrous Lactose monohydrate, Mannitol, Magnesium carbonate, Magnesium oxide, Microcrystalline cellulose, Sorbitol, Starch, micro-silica gel and talcum powder and lubricant is selected from magnesium stearate, glyceryl monostearate, stearic acid, talc, DMSO, Tween 80, Normal saline 0.9% sodium chloride and the like.

11. The composition as claimed in claim 8, wherein the compound of Formula 1 is an imidazole glycerol phosphate dehydratase (IGPD) inhibitor.

12. A method for inhibiting Mycobacterium tuberculosis, wherein said method comprises administering a therapeutically effective amount of a compound with Formula 1 to a subject in need thereof.

13. The method as claimed in claim 12, wherein the subject is an animal selected from the group comprising human, canine, feline, bovine, ovine, caprine, porcine, avian, piscine, and equine species.

14. The method as claimed in claim 12, wherein the compound is administered to the subject in a dose in the range of 5 mg/kg to 5000 mg/kg body weight.

15. The method as claimed in claim 12, wherein the compound is administered to the subject by oral administration, parenteral administration or topical administration.

16. The method of claim 12, further comprising administering one or more other antibacterial agents.

17. The method of claim 16, wherein the one or more other antibacterial agents are selected from rifampicin, isoniazid, pyrazinamide, amikacin, ethionamide, ethambutol, streptomycin, para-aminosalicylic acid, cycloserine, kanamycin, thioacetazone delamanid, moxifloxacin, gatifloxacin, ofloxacin, ciprofloxacin, sparfloxacin, clarithromycin, amoxycillin, rifamycins, rifabutin, rifapentine, or a combination thereof.

18. The method of claim 12, wherein the one or more other antibacterial agents and the compound with Formula 1 are administered simultaneously, separately or sequentially.
